(19) United States
(12) Patent Application Publication
(10) Pub. No.: US 2009/0298101 A1
(43) Pub. Date: Dec. 3, 2009

(54) Blood C5a Levels as an Indicator of Rhinoconjunctivitis Severity

(75) Inventor: Rauno Joks, Port Washington, NY (US)

Correspondence Address: SCULLY, SCOTT, MURPHY & PRESSER, P.C., 400 GARDEN CITY PLAZA, SUITE 300, GARDEN CITY, NY, 11530, US

(73) Assignee: The Research Foundation of State University of New York, Albany, NY (US)

(21) Appl. No.: 11/884,585

(22) PCT Filed: Feb. 16, 2006
(86) PCT No.: PCT/US2006/005354

§ 371 Date: Jun. 26, 2009

Related U.S. Application Data
(60) Provisional application No. 60/654,533, filed on Feb. 18, 2005.

(51) Int. Cl. G01N 33/566

(52) U.S. Cl. 435/7.91; 435/7.92

(57) ABSTRACT

The present invention provides methods of determining the severity of rhinoconjunctivitis in a patient by determining the levels of C5a or C5a-desArg in the patient's blood, plasma or serum.

### FIELD OF THE INVENTION

This invention relates to methods for determining rhinoconjunctivitis severity. More specifically, the present invention relates to methods of determining the severity of rhinoconjunctivitis in a patient by determining the C5a level in the patient's blood.

### BACKGROUND OF THE INVENTION

Rhinitis is an inflammation of the nasal mucosa, often due to an allergic reaction to pollen, dust or other airborne substances (allergens). Although the pathophysiology of many types of rhinitis is unknown, an accurate diagnosis is necessary, since not all types of rhinitis will respond to the same treatment measures.

Chronic rhinitis includes Atopic Rhinitis, Seasonal Allergic Rhinitis (also known as hay fever), Perennial Rhinitis (year-round) with Allergic Triggers, Perennial Rhinitis with Non-Allergic Triggers, Idiopathic Non-Allergic Rhinitis, Infectious Rhinitis, Rhinitis Medicamentosa, Mechanical Obstruction, Hormonal and other types of rhinitis.

Allergic (seasonal and perennial) rhinitis is the most common of all atopic diseases in the United States, affecting about 10 to 25% of the adult population, which is characterized by an inflammation of the nasal mucous membranes due to an allergic response. The main causes of seasonal allergic rhinitis are tree, grass or weed pollen. While no one dies directly as a result of allergic rhinitis, the economic impact is substantial. Over $600 million is spent in the USA annually in the management of this disease. This does not include the costs of the 2 million lost workdays, 3 million lost school days and 28 million days of decreased productivity from the symptoms of the disease and/or side effects of the medications used to treat them.

Clinically, information is gained from a nasal examination which may reveal pale, boggy turbinate as well as clear to greenish rhinorrhea. When colored nasal secretions are stained and examined, they typically reveal large numbers of eosinophils as the main inflammatory cell. In many instances (particularly in children) complications such as chronic otitis media, rhinosinusitis and conjunctivitis can be traced to chronic obstruction from allergic rhinitis. See http://www.hon.ch/Library/Theme/Allergy.

Conjunctivitis (Pink Eye) is an inflammation of the conjunctiva, a membrane that lines the inside of the eyelid and touches the white part of the eye, secreting a mucous that lubricates the eyeballs. There are many different causes of conjunctivitis. The main causes are infectious, which result from bacterial or viral infections, and noninfectious, which is due to certain allergies (such as pollen or animal dander) and chemical irritants (such as smoke, preservatives in contact lens solutions and some eye drops, or the chlorine in swimming pools). Allergic Conjunctivitis is usually accompanied by intense symptoms (itching, redness, tearing, and swelling of the eye membranes). It is frequently seasonal, and is accompanied by other typical allergic symptoms such as sneezing, itchy nose, or scratchy throat. See http://www.hon.ch.

Rhinoconjunctivitis is a combination of rhinitis and conjunctivitis. There is currently no blood test for assessing rhinoconjunctivitis symptom severity.

Activation of the classical, lectin complement pathways can result in proteolytic cleavage of C5 to two fragments, C5a and C5b, both of which can stimulate cytokine production. As part of a hemolytically active membrane attack complex, C5b causes signaling in neutrophils and endothelia, inducing chemokine production by the latter (Wang et al., Blood 85: 2570-2578, 1995; Wang et al., J. Immunol. 156: 786-792, 1996; Kilgore et al., Am J. Pathol. 150: 2019-2031, 1997). C5a has pleiotropic effects on inflammation, being chemotactic for all myeloid lineages, inducing degranulation and the production of a variety of proinflammatory mediators by granulocytes and increasing vascular permeability (Gerard et al., Annu. Rev. Immunol. 164: 3009-3017, 2000). C5a also stimulates monocyte and macrophage production of the proinflammatory cytokines TNF-α, IL-1 and IL-6 (Morgan et al., J. Immunol. 148: 3937-3942, 1992; Schindler et al., Blood 76: 1631-1638, 1990; Cavaillon et al., Eur. J. Immunol. 20: 253-257, 1990). Inhibition of stimulation of monocytes and macrophages by C5a through the C5a receptor has resulted in the inhibition of production of IL-12 (Karp, Nature Immun., 2000), a Th1 promoting cytokine, by these cells.

The nascent C5a fragment of C5, once formed in blood plasma or serum, is rapidly cleaved to the C5a-desArg form by the endogenous serum carboxypeptidase N enzyme (Bokisch et al. J. Clin. Invest. 49: 2427-36, 1970).

Prior to the present invention, there was no recognition that the plasma levels of C5a or levels of C5a-desArg in rhinoconjunctivitis patients correlate with the severity of rhinoconjunctivitis. The present invention, for the first time, recognizes the significant correlation between plasma level of C5a or C5a-desArg and severity of rhinoconjunctivitis, which can be used as a tool to monitor symptom severity and/or treatment response.

### SUMMARY OF THE INVENTION

The present inventor has unexpectedly discovered that the plasma C5a levels in rhinoconjunctivitis patients correlate with the severity of rhinoconjunctivitis determined by using conventional clinical criteria.

Accordingly, in one aspect, the present invention provides a method of determining the severity of rhinoconjunctivitis in a patient by detecting the level of C5a or C5a-desArg in a blood, plasma or serum sample from the patient.

In another aspect, the present invention provides a method of determining the severity of rhinoconjunctivitis in a patient by detecting the level of C5a or C5a-desArg in a blood, plasma or serum sample from the patient, and correlating the level with a rhinoconjunctivitis severity score.

In one aspect, the present invention provides a method of determining the severity of rhinitis in a patient comprising detecting the level of C5a or C5a-desArg in the blood of said patient.

In another aspect, the present invention provides a method of determining the severity of rhinitis in a patient by detecting the level of C5a or C5a-desArg in a blood, plasma or serum sample from the patient, and correlating the level with a rhinitis severity score.

In still another aspect, the present invention provides a method of determining the severity of conjunctivitis in a patient comprising detecting the level of C5a or C5a-desArg in the blood of said patient.

In yet another aspect, the present invention provides a method of determining the severity of conjunctivitis in a patient by detecting the level of C5a or C5a-desArg in a blood, plasma or serum sample from the patient, and correlating the level with a conjunctivitis severity score.

### BRIEF DESCRIPTION OF THE DRAWINGS

FIG. 1 depicts the association of plasma C5a-desArg level with rhinoconjunctivitis severity scores determined using conventional clinical criteria.

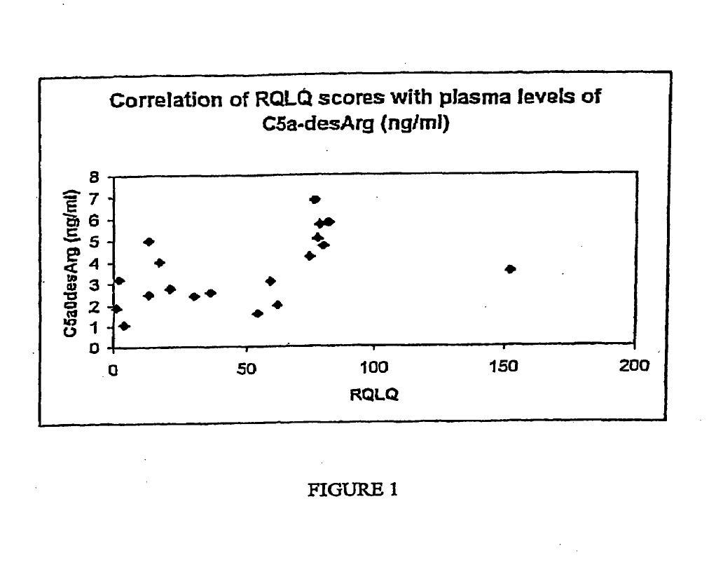

### DETAILED DESCRIPTION OF THE INVENTION

The present inventor has surprisingly found that blood C5a-desArg levels in rhinoconjunctivitis patients correlate with the severity of rhinoconjunctivitis determined by using conventional clinical criteria. Accordingly, the present invention provides, for the first time, a blood-based test method for determining the severity of rhinoconjunctivitis in a patient.

In one embodiment, the present invention provides a method for determining the severity of rhinoconjunctivitis in a patient by detecting the level of C5a or C5a-desArg in the patient's blood sample.

By “blood sample” is meant a whole blood sample, a plasma sample or a serum sample.

To detect the blood level of C5a or C5a-desArg in a patient, a blood sample is taken from the patient. The blood sample can be a sample of whole blood drawn from the patient, or a sample of the serum or plasma portion derived from whole blood of the patient. Methods for obtaining the plasma or serum portion of whole blood are well known in the art and are also illustrated in Example 2, provided hereinbelow.

Detection of the levels of C5a or C5a-desArg in patients' blood can be carried out by using antibodies specific for C5a or C5a-desArg in any enzyme-immunological or immunochemical detection format, such as ELISA (enzyme linked immunosorbent assay), EIA (enzyme immunoassay), RIA (radioimmunoassay), Western Blot analysis, DIPSTICK and the like. Depending upon the assay used, the blood samples or the antibodies can be labeled by an enzyme, a fluorophore or a radioisotope. See, e.g., Coligan et al. Current Protocols in Immunology, John Wiley & Sons Inc., New York, N.Y. (1994); and Frye et al., Oncogen 4: 1153-1157, 1987. Preferably, the detection is carried out using an ELISA assay where labeled antibodies against C5a-desArg are immobilized, as exemplified in Example 2 hereinbelow.

In another embodiment, the present invention provides a method of determining the rhinoconjunctivitis severity of a patient by detecting the level of C5a or C5a-desArg in a blood, plasma or serum sample from the patient, and correlating such level with a rhinoconjunctivitis severity score. As discovered by the present inventors, blood levels of C5a or C5a-desArg in rhinoconjunctivitis patients correlate with rhinoconjunctivitis severity scores determined using conventional clinical criteria. Conventional clinical criteria used in determining rhinoconjunctivitis severity scores are described in Rhinoconjunctivitis Quality of Life Questionnaire (RQLQ) (QOL Technologies, Ltd., 1996, incorporated herein by reference) (“RQLQ”). According to the present invention, a patient's rhinoconjunctivitis severity score is established from the patient's response to self-administered questions as provided in RQLQ. The patient is asked as to how he/or she has been troubled, particularly, by his/her nose/eye symptoms, during past week in associated with his/her activities, sleep, non-nose/eye symptoms, practical problems, nasal problems, eye symptoms, and emotion. The rhinoconjunctivitis severity score can be calculated based on the responses to RQLQ by methods known in the art. For example, the severity score can be a sum of the numerical responses to the twenty eight questions in the survey of RQLQ.

According to the present invention, once the level of C5a or C5a-desArg in a patient's blood is determined, such level can be compared to a predetermined value of C5a or C5a-desArg levels, or preferably, to a set of predetermined values of C5a or C5a-desArg levels, where each predetermined value corresponds to a rhinoconjunctivitis severity score determined based on conventional clinical criteria.

In one embodiment, the present invention provides a method of determining the severity of rhinitis in a patient comprising detecting the level of C5a or C5a-desArg in the blood of said patient.

In another embodiment, the present invention provides a method of determining the severity of rhinitis in a patient by detecting the level of C5a or C5a-desArg in a blood, plasma or serum sample from the patient, and correlating the level with a rhinitis severity score.

In still another embodiment, the present invention provides a method of determining the severity of conjunctivitis in a patient comprising detecting the level of C5a or C5a-desArg in the blood of said patient.

In yet another embodiment, the present invention provides a method of determining the severity of conjunctivitis in a patient by detecting the level of C5a or C5a-desArg in a blood, plasma or serum sample from the patient, and correlating the level with a conjunctivitis severity score.

The present invention is further illustrated by the following non-limiting examples.

### Example 1

### Determination of Plasma C5a-desArg Levels

Plasma was obtained from blood drawn on patients on a single visit. The patients were seen regularly in the Asthma Center Of Excellence at State University of New York at Brooklyn, N.Y. In addition to review of rhinoconjunctivitis symptoms, the patients were also clinically assessed for presence and degree of rhinoconjunctivitis as well as allergen sensitization (by skin prick testing). At a later time, the patients' rhinoconjunctivitis severity scores were determined using standardized criteria based on the questionnaire provided by Rhinoconjunctivitis Quality of Life Questionnaire (RQLQ) (QOL Technologies, Ltd., 1996). Plasma C5a-desArg levels were determined by using the OptEIA™ human C5a kit from PHARMINGEN (a division of Becton, Dickinson and Company, 10975 Torregyana Road, San Diego, Calif. 92121) and following the manufacturer's instructions (provided in Example 2).

### Example 2

### The OptEIA™ Human C5a Test

### Principle of the Test

The OptEIA™ ELISA test is a solid phase sandwich ELISA (Enzyme-Linked Immunosorbent Assay). It utilizes monoclonal antibody specific for human C5a-desArg coated on a 96-well plate. Standards and samples are added to the wells, and any C5a-desArg present binds to the immobilized antibody. The wells are washed and a mixture of biotinylated polyclonal anti-human C5a antibody and avidin-horseradish peroxidase is added, producing an antibody-antigen-antibody “sandwich”. The wells are again washed and a substrate solution is added, which produces a blue color in direct proportion to the amount of C5a-desArg present in the initial sample. The Stop Solution changes the color from blue to yellow, and the wells are read at 450 nm.

### Reagents Used:

Antibody Coated Wells: 1 plate of 96 breakable wells (12 strips×8 wells) coated with anti-human CSa-desArg monoclonal antibody.
Detection Antibody: 15 ml of biotinylated anti-human C5a polyclonal antibody with 0.15% ProClin-150 as preservative.
Standards: 3 vials lyophilized human serum containing a defined amount of C5a-desArg (quantity as noted on vial label).
Enzyme Concentrate (250×): 150 μl of 250× concentrated Avidin-horseradish peroxidase conjugate with 0.01% thimerosal as preservative.
Standard/Sample Diluent: 15 ml of animal serum with 0.09% sodium azide as preservative.
ELISA Diluent: 6 ml of a buffered protein base with 0.09% sodium azide as preservative.
Wash Concentrate (20×): 100 ml of 20× concentrated detergent solution with 0.02% thimerosal as preservative.
TMB One-Step Substrate Reagent: 15 ml of 3,3′,5,5′ tetramethylbenzidine (TMB) in buffered solution.
Stop Solution: 13 ml of 1M phosphoric acid.
Plate Sealers: 2 sheets with adhesive backing.

### Storage

Keep the unopened kits at 2-8° C. Before use, bring all reagents to room temperature (18-25° C.). Immediately after use, return to proper storage conditions. Lyophilized standards are stable until kit expiration date. After reconstitution, use freshly reconstituted standard within 12 hours (stored at 2-8° C.).

### Specimen Collection and Handling

Specimens should be clear, non-hemolyzed and non-lipemic. It is recommended that normal human EDTA plasma samples be used undiluted, i.e., neat in this assay. Samples with expected values higher than the top standard, 40 ng/ml, should be diluted with Standard/Sample Diluent prior to running the assay. All specimen handling operations should be carried out at 4° C. for plasma and for serum (immediately after clotting).

Plasma: Collect plasma using disodium EDTA as the anticoagulant. If possible, collect the plasma into a mixture of disodium EDTA and Futhan⁵ to stabilize the sample against spontaneous in vitro complement activation. Immediately centrifuge samples at 4° C. for 15 minutes at 1000×g. Assay immediately or store samples on ice for up to 6 hours before assaying. Aliquots of plasma may also be stored at −70° C. for extended periods of time. Avoid repeated freeze-thaw cycles.

Serum: Use a serum separator tube and allow samples to clot for 60±30 minutes. Centrifuge the samples at 4° C. for 10 minutes at 1000×g. Remove serum and assay immediately or store samples on ice for up to 6 hours before assaying. Aliquots of serum may also be stored at −70° C. for extended periods of time. Avoid repeated freeze-thaw cycles.

Other biological samples: Remove any particulate matter by centrifugation and assay immediately or store samples at −70° C. Avoid repeated freeze/thaw cycles.

### Reagent Preparation

1. Bring all reagents to room temperature (18-25° C.) before use.

### 2. Standards:

a. Reconstitute 1 vial lyophilized Standard with required volume (noted on vial label) of Standard Diluent to prepare a 40 ng/ml stock standard. Allow the standard to equilibrate for at least 15 minutes before making dilutions. Vortex to mix.
b. Add 300 μl Standard Diluent to 6 tubes. Label as 20 ng/ml, 1.25 ng/ml, and 0.625 ng/ml.
c. Perform serial dilutions by adding 300 μl of each standard to the next tube and vortexing between each transfer. The undiluted standard serves as the high standard (40/ng/ml). The Standard Diluent serves as the zero standard (ng/ml).

### 3. Working Detector

See Assay Procedure (below), step 5.

### 4. Wash Buffer

If the Wash Concentrate contains visible crystals, warm to room temperature and mix gently until dissolved. Dilute required quantity of 20× Wash Concentrate with deionized or distilled water, mix. (To prepare 2.0 L, add 100 ml Wash Concentrate to 1900 ml water. At least 500 ml solution should be prepared for a full 96-well plate).

### 5. TMB One-Step Substrate Reagent

No more than 15 minutes prior to use, add required volume of TMB One-Step Substrate Reagent to a clean tube or reservoir. To prevent contamination, pipette out from the tube/reservoir instead of directly from bottle. Avoid prolonged exposure to light or contact with metal, air, or extreme temperature as color may develop.

### Assay Procedure

1 Bring all reagents and samples to room temperature (18-25° C.) prior to use. It is recommended that all standards and samples be run in duplicate. A standard curve is required in each assay run.
2. Place required quantity of test strips/wells in well holder. Wells are provided in breakable 8-well strips. Strips may be “broken” into individual wells, replaced in well holder, and assayed. Return any unused wells to sealed pouch for 2-8° C. storage.
3. Pipette 50 μl of ELISA Diluent into each well.
4. Pipette 100 μl of each standard (see Reagent Preparation, step 2) and sample into appropriate wells. Gently shake/tap the plate for 5 seconds to mix. Cover wells with Plate Sealer and incubate for 2 hours at room temperature.
5. Prepare Working Detector. Within 15 minutes prior to use, pipette required volume of Detection antibody into a clean tube or flask. Add in required quantity of Enzyme Concentrate (250×), vortex or mix well. For a full 96-well plate, add 48 μl of Enzyme Concentrate into 12 ml of Detection Antibody.
6. Decant or aspirate contents of wells. Wash wells by filling with at lest 300 μl/well prepared Wash buffer (see Reagent Preparation, step 4) and then decanting/aspirating. Repeat wash 4 times for a total of 5 washes. After the last wash, blot plate on absorbent paper to remove any residual buffer. Complete removal of liquid is required for proper performance.
7. Add 100 μl of prepared Working Detector (see step 5 above) to each well. Gently shake/tap the plate for 5 seconds to mix. Cover wells with plate Sealer and incubate for 1 hour at room temperature.
8. Wash wells as in Step 6, but a total of 7 times. In this final wash step, soak wells in wash buffer for 30 seconds to 1 minute for each wash. Thorough washing at this step is very important.
9. Add 100 μl of TMB One-Step Substrate Reagent to each well. Gently shake/tap the plate for 5 seconds to mix. Incubate plate (without Plate Sealer) for 30 minutes at room temperature in the dark.
10. Add 50 μl of Stop Solution to each well. Gently shake/tap the plate for 5 seconds to mix.
11. Read absorbance at 450 nm within 30 minutes of stopping reaction. If wavelength correction is available, subtract A (570 nm) from A (450 nm).

### Calculation of Results

Calculate the mean absorbance for each set of duplicate standards, controls and samples. Subtract the mean zero standard absorbance from each. Plot the standard curve on log-log graph paper, with C5a-desArg concentration on the x-axis and absorbance on the y-axis. Draw the best fit straight line through the standard points.

To determine the C5a-desArg concentration of the unknowns, find the unknowns' mean absorbance value on the y-axis and draw a horizontal line to the standard curve. At the point of intersection, draw a vertical line to the x-axis and read the C5a-desArg concentration. If samples were diluted, multiply the C5a-desArg concentration by the dilution factor. Computer curve-fitting statistical software may also be employed.

### Example 3

### Relations of Plasma C5a Levels to Allergic Disease in Pediatric and Adult Cohorts

Rationale. Murine C5 deficiency has been linked to allergen-induced airway hyperreactivity (Karp, 2000). This deficiency has not been studied in humans. The present studies investigated the relationship of plasma levels of C5a-desArg and other complement split products (CSP) to clinical allergic disease in both pediatric and adult populations.
Method. Plasma levels of C5a-desArg, C4a-desArg and C3a-desArg (ELISA) were measured from allergic children (n=18) (no immunotherapy) and allergic adults (n=19) (most of whom were receiving immunotherapy). Asthma severity scores (0-4) were assigned according to 2002 NHLBI criteria. Additional data for the adult group included: rhinitis and asthma symptoms scores (RQLQ and AQLQ, Juniper), spirometry, exhaled nitric oxide (eNO), CBC, and T cell studies.
Results. For the pediatric clinic group, there was a significant inverse correlation between plasma C5a levels and asthma severity (p=0.038), but not to IgE (p=0.46). No correlation was found between severity and C4a, C3a, or IgE (p=0.20, 0.66, and 0.61, respectively). Increased adult asthma severity scores correlated with increased C5a (p=0.02). In addition, increased rhinoconjunctivitis and asthma symptoms correlated with increased C5a levels (p=0.008 and p=0.0065, respectively). No other parameter correlated with C5a. C4a levels did not correlate with any clinical or laboratory parameters.
Conclusions. Plasma C5a levels correlated with severity of rhinoconjunctivitis. Increased C5a associated with decreased asthma severity in allergic children illustrates that a protective stimulus by C5a can be active at young ages, possibly through IL-12 levels. Long term allergen exposure in adults, including by immunotherapy, can increase anaphylatoxin activity and increases allergic responses to C5a.

TABLE 2
Correlation of Plasma C5a-desArg levels with
rhinoconjunctivitis clinical severity

| | Rhinoconjunctivitis | Plasma C5a-desArg |
|---|---|---|
| Subject | Severity Score (RQLQ) | (ng/ml) |
| MA | 152 | 3.522 |
| SA | 78 | 5.041 |
| PS | 77 | 6.812 |
| DH | 13 | 4.976 |
| MK | 54 | 1.56 |
| AM | 17 | 3.987 |
| DS | 2 | 3.187 |
| rj | 1 | 1.856 |
| csf | 59 | 3.075 |
| aa | 75 | 4.205 |
| wk | 4 | 1.063 |
| IR | 36 | 2.562 |
| IS | 13 | 2.473 |
| vc | 79 | 5.683 |
| dz | 30 | 2.392 |
| cb | 80 | 4.71 |
| mb | 21 | 2.75 |
| yb | 82 | 5.77 |
| dn | 62 | 1.944 |

TABLE 3
Spearman Correlation Coefficients of C5a and
C4a with clinical and laboratory parameters

| | C5a | C4a |
|---|---|---|
| RQLQ | 0.0078 | 0.95 |
| Asthma AQLQ | 0.0065 | 0.17 |
| Asthma severity | 0.0233 | 0.52 |
| IgE | 0.8473 | 0.71 |
| Nitric Oxide (PPB) | 0.3283 | 0.69 |
| FEV1/FEV | 0.5638 | 0.31 |
| FEV1 | 0.8277 | 0.54 |
| PEF (peak flow) | 0.1723 | 0.47 |
| PEF 25-75 | 0.4949 | 0.83 |
| PMN | 0.5293 | 0.48 |
| CD4/CD8 | 0.4609 | 0.86 |

### Example 4

### Change in Plasma C5a Levels after Allergen Immunotherapy Correlates with Skin Reactivity, Rhinitis Symptoms, and Dust Mite Dosage

RATIONALE: Complement split product C5a is increased in BAL fluid after segmental allergen provocation. C5a is also generated in vitro in serum in the presence of allergens. This study investigated if plasma C5a levels are altered after routine allergen immunotherapy (IT) for rhinitis/asthma.
METHODS: C5a-desArg levels in plasma (ELISA) from IT subjects (n=16) were determined immediately before and 1 or after IT. Skin reactivity to each allergen (in duration) was determined at 15 min and 1 hr. Spirometry was performed before and at 1 hr. Rhinoconjunctivitis quality of life symptom scores (RQLQ, Juniper) and total serum IgE level were determined.
RESULTS: Change in plasma C5a levels was found to correlate with change in total mean wheal diameter (p=0.05), total dust mite IT dose (p=0.04), and total RQLQ score (p=0.03). Change in C5a did not correlate with total IT dose or IgE. Although change in C5a levels did not correlate with spirometric parameters at 1 hr, there was a significant negative correlation between total mean wheal diameter change and change in peak flow (PEFR)(p=0.005). The mean wheal diameter induced by dust mite IT was negatively correlated with change in FEF25-75 (p=0.007), but not PEFR (p=0.4).
CONCLUSIONS: The correlation of change in C5a levels with dust mite dosage, changes in wheal size, and symptoms scores strongly suggests that C5a is crucial in the maintenance of allergic inflammation.

### Example 5

### Cutaneous Allergic Inflammation Resulting from Allergen Immunotherapy is Associated with Increase in Plasma Levels of C5a and C3a and Altered Spirometric Outcome

This study investigated the levels of complement split products C5a desArg and C3a desArg (CSP) in blood after routine allergen immunotherapy for asthma (AS) and rhinoconjunctivitis (RC).

Immunotherapy was given to allergic adults (n=5, AS and RC; n=11, RC alone). Plasma CSP and IgE levels were determined before (CSP, IgE), and at 1 and 3 hrs after therapy (CSP) (ELISA, microimmunofluorimetry). Wheal induration was determined at 15 min, and at 1 and 3 hr. Spirometry was performed before and at 1 and 3 hr. RC and AS quality of life symptom scores (RQLQ and AQLQ, Juniper) were obtained. Changes in plasma C5a levels at 0 to 1 hr correlated with changes in: total mean wheal diameter (p=0.05), total RQLQ (p=0.03), and total mean dose of dust mite allergen (DM) (p=0.04). At 1 to 3 hr there was a negative correlation between change in C5a and DM dose (p<0.01). There was no correlation between change in C5a and DM dose (P<0.01). There was no correlation between change in C5a and total wheal diameter at 0 to 3 hr. Change in C5a levels did not correlate with IgE levels. Change in C3a levels never correlated with change in total mean wheal diameter or total RQLQ. At 0 to 3 hr there was a significant correlation between change in C3a and change in wheal in duration from. DM immunotherapy (p=0.01). Changes in C3a at 1 to 3 hr were associated with higher levels of IgE (p=0.04). Spirometric changes correlated with cutaneous reactivity to allergen therapy. At 1 and 3 hr, there was a negative association between change in total mean wheal diameter and peak flow rate (p<0.01 and p=0.01, respectively). Increase in mean wheal diameter from DM therapy was associated with decrease in FEF25-75 at 1 hr (p<0.01), but not at 3 hr. Changes in CSP levels did not correlate with changes in spirometry.

These results suggest that plasma levels of CSP, especially C5a, are useful as biomarkers of the allergic inflammation underlying allergic rhinoconjunctivitis.

We claim:

1. A method of determining the severity of rhinoconjunctivitis in a patient comprising detecting the level of C5a or C5a-desArg in the blood of said patient.

2. The method of claim 1, wherein the determination is made by obtaining a blood sample from said patient, and detecting the level of C5a or C5a-desArg in said sample.

3. The method of claim 2, wherein said sample is selected from a whole blood sample, a serum sample or a plasma sample.

4. The method of claim 2, wherein the level of C5a or C5a-desArg in said sample is detected by an immunological assay.

5. The method of claim 4, wherein said immunological assay is an assay selected from ELISA (enzyme linked immunosorbent assay), EIA (enzyme immunoassay), RIA (radioimmunoassay), or Western Blot analysis.

6. The method of claim 1, further comprising correlating said level to a rhinoconjunctivitis severity score.

7. The method of claim 6, wherein the detection is made by obtaining a blood sample from said patient, and detecting the level of C5a or C5a-desArg in said sample.

8. The method of claim 7, wherein said sample is selected from a whole blood sample, a serum sample or a plasma sample.

9. The method of claim 7, wherein the level of C5a or C5a-desArg in said sample is detected by an immunological assay.

10. The method of claim 9, wherein said immunological assay is an assay selected from the group consisting of ELISA (enzyme linked immunosorbent assay), EIA (enzyme immunoassay), RIA (radioimmunoassay), and Western Blot analysis.

11. A method of determining the severity of rhinitis in a patient comprising detecting the level of C5a or C5a-desArg in the blood of said patient.

12. The method of claim 11, further comprising correlating said level to a rhinitis severity score.

13. A method of determining the severity of conjunctivitis in a patient comprising detecting the level of C5a or C5a-desArg in the blood of said patient.

14. The method of claim 1, further comprising correlating said level to a conjunctivitis severity score.
